(19) United States
(12) Patent Application Publication
(10) Pub. No.: US 2006/0068503 A1
(43) Pub. Date: Mar. 30, 2006

(54) Solid support membranes for ion channel arrays and sensors: application ro rapid screening of pharmacological compounds

(76) Inventor: John Cuppoletti, Cincinnati, OH (US)

Correspondence Address: DINSMORE & SHOHL, LLP, 1900 CHEMED CENTER, 255 EAST FIFTH STREET, CINCINNATI, OH, 45202, US

(21) Appl. No.: 11/224,422

(22) Filed: Sep. 12, 2005

Related U.S. Application Data
(60) Provisional application No. 60/608,764, filed on Sep. 10, 2004.

(51) Int. Cl. G01N 33/543

(52) U.S. Cl. 436/518

(57) ABSTRACT

The present invention relates to synthetic membranes and provides a solid supported membrane comprising: a support; a chromium layer; a gold layer; an alkyl monolayer, a lipid monolayer; and an ion channel protein, wherein the alkyl monolayer and the lipid monolayer form a bilayer, and further wherein a functionally reconstituted ion channel is supported, and methods for forming them. The invention further provides methods and applications of the membranes in rapid screening for pharmacological agents and as bioelectric smart sensors.

### RELATED APPLICATIONS

This application claims priority under 35 U.S.C. §119 to U.S. Provisional Application Ser. No. 60/608,764, filed on Sep. 10, 2004.

### GOVERNMENT INTERESTS

This invention was made, at least in part, with funds from the Federal Government, awarded through awarded through MURI grant number DAAD 19-0201-0227 ARMY. The U.S. Government therefore has certain acknowledged rights to the invention.

### FIELD OF THE INVENTION

The present invention relates to the field of synthetic membranes, and, specifically, to reconstitution of functioning ion channel proteins on solid supported membranes. In particular, the present invention provides a solid supported membrane (SSM) comprising a functionally reconstituted ion channel protein, the use of such membranes to rapid screening of pharmacological reagents affecting ion channel function, and applications in the formation of membranes useful as bioelectric sensors in, for example, industrial applications.

### BACKGROUND OF THE INVENTION

The use of solid supported membranes has become a popular method of studying biological processes of cellular proteins.

It has been reported that some proteins behave similarly to their cellular counterparts when reconstituted on a solid supported membrane. For example, recent studies on the charge translocation by the Na/K ATPase on a solid supported membrane, cytochrome b5 on a cushioned solid supported membrane, and rhodopsin on a solid supported membrane to study transduction activation demonstrated similarity in behavior of these proteins on solid supported surfaces compared to that measured in biological membranes.

The main advantages of using solid supported membranes include their mechanical stability and ability to rapidly change the solution environment.

However, it is the belief of the present inventor that reconstitution of functional ion channels on SSMs has not been achieved prior to the studies undertaken thereby. Ion channels are expected to act similarly to the previously reported behavior of other biological proteins on solid supported membranes.

Some pharmacological inhibitors and other effectors of particular ion channels are known, and, in some cases, their effects on ion channel-mediated currents in cells have been highly studied. Methods known in the art for determining the effects of pharmacological agents on ion channels have relied on assays based on indirect measurement, which require the use of intact cells and methods that are often not easily interpretable or are not sufficiently sensitive and/or accurate. Furthermore, the planar lipid-bilayers and the patch clamp approaches known in the art and conventionally utilized, lack stability and size, which prevent the membrane from being reacted with multiple substances, washed and even re-used for multiple cycles. This severely limits their usefulness in rapid or high-throughput methods for screening pharmacological compounds.

It would be advantageous to reconstitute channels on SSMs, expose the channels to pharmacological compounds, and measure current through the channel in the presence of those compounds relative to those measured in response to holding potential.

The strength and stability of SSMs comprising reconstituted functional ion channels makes them desirable for use as screening devices for pharmacological agents affecting ion channels.

### SUMMARY OF THE INVENTION

Accordingly, the present invention provides solid supported membranes (SSM) comprising functional reconstituted ion channels. The present invention also provides methods of using the SSM bound ion channels to screen pharmacological reagents affecting ion channel function, and applications directed to bioelectric sensor technology.

One embodiment of the invention is directed to a solid supported membrane comprising: a support; a chromium layer; a gold layer; an alkyl monolayer, a lipid monolayer; and an ion channel protein, wherein the alkyl monolayer and the lipid monolayer form a bilayer, and further wherein a functionally reconstituted ion channel is supported.

Another embodiment provides an assay for screening compounds to identify pharmacological agents affecting an ion channel. The assay comprises: (a) providing a solid supported membrane comprising: a support; a chromium layer; a gold layer; an alkyl monolayer, a lipid monolayer; and an ion channel protein, wherein the alkyl monolayer and the lipid monolayer form a bilayer, and further wherein a functionally reconstituted ion channel is supported; (b) contacting (a) with a solution comprising an ion; (c) applying a potential and obtaining a stable ion current; (d) contacting the solid support membrane with a compound to be identified; and (e) measuring a change in the ion current.

A further embodiment is directed to a method for forming a functionally reconstituted ion channel on a solid supported membrane. The method comprises the steps of: a. providing a solid support coated with a chromium layer and a gold layer; b. forming an alkyl-thiol monolayer on the gold layer; c. depositing a lipid monolayer on the alkyl-thiol monolayer to form a bilayer; and d. contacting the bilayer with an ion channel protein.

A particular embodiment is directed to a bioelectric sensor comprising a solid supported membrane comprising: a support; a chromium layer; a gold layer; an alkyl monolayer, a lipid monolayer; and an ion channel protein, wherein the alkyl monolayer and the lipid monolayer form a bilayer, and further wherein a functionally reconstituted ion channel is supported on the solid supported membrane. These, and other embodiments of the present invention, are more fully expounded upon and set forth in detail below.

### DETAILED DESCRIPTION OF THE INVENTION

A solid supported membrane is a synthetic membrane constructed on, for example, a silanized glass slide, coated with a thin layer of chromium and then gold. The gold surface is treated to provide a bonding moiety, with, for example, a long chain alkyl thiol, which is subsequently coated with a lipid monolayer. The bilayer formed by the lipid and the alkyl chain on the gold surface is similar to the planar lipid bilayer widely used to study ion channel activity, but it is much more stable. It has been reported that some proteins behave similarly to their cellular counterparts when reconstituted on a solid supported membrane. In addition, recent studies on the charge translocation by the Na/K ATPase on a solid supported membrane, cytochrome b5 on a cushioned solid supported membrane, and rhodopsin on a solid supported membrane to study transduction activation demonstrated similarity in behavior of these proteins on solid supported surfaces compared to that measured in biological membranes.

The main advantages of using solid supported membranes include their mechanical stability and ability to rapidly change the solution environment. Ion channels are expected to act similarly to the previously reported behavior of other biological proteins on solid supported membranes. The voltage gated Kv1.5 K+ channel is typical of ion channels. It is found in human and mammalian cardiovascular cells. Kv1.5 is a delayed rectifier that controls the membrane potential of neurons and its biological activity in cells has been studied extensively. In addition, this particular ion channel has been investigated in detail. Inhibitors and other effectors of Kv1.5 channels are available, and their effects on Kv1.5-mediated K+ currents in cells have been highly studied.

Two pharmacological reagents applied to Kv1.5 K+ channels reconstituted on SSM exhibited similar inhibitory effects as those measured using Kv1.5 in biological membranes. SSM mounted ion channels were stable enough to be washed with buffer solution and reused many times, allowing solution exchange essential for pharmacological drug screening.

One embodiment is directed to a solid supported membrane (SSM) comprising a functionally reconstituted ion channel. The SSM comprises: a support; a chromium layer; a gold layer; an alkyl monolayer, a lipid monolayer; and an ion channel protein, wherein the alkyl monolayer and the lipid monolayer form a bilayer, and further wherein a functionally reconstituted ion channel is supported.

The cells containing ion channels may come from any species and contain either wild type or recombinant native, engineered proteins, or synthetic ion channels. The membrane fragments may be derived from membranes that are unpurified or purified by any of a variety of biochemical techniques. Furthermore, the membranes may be either closed (vesicles), detergent solutions, micelles, or broken sheets of cells. Organic and fluorocarbon solutions of ion channels may also be used. In a specific embodiment of the solid supported membrane, the ion channel protein comprises native protein, recombinant protein, or combinations thereof, and in another more specific embodiment the ion channel comprises a Kv1.5K⁺⁺ channel.

One skilled in the art will appreciate that the layers of the solid-supported membrane may comprise various thicknesses. Furthermore, as the underlying structures of the solid-supported membrane do not contribute to the processes leading to insertion of the ion channel into the membrane, supports comprising various materials may be used to support the bilayers with ion channels. Other support material that may be suitable and includes, but is not limited to, agar polymers, conducting polymers, glass, silinated glass, alkylated hydrogels, plastics, MYLAR, metallic substrates, or combinations thereof. In a specific embodiment, the solid support comprises silinated glass.

In one particular embodiment, the voltage gated Kv1.5 K+ channel is reconstituted on a solid supported membrane. As demonstrated by the Example below, the present inventors conducted a study whereby a measured current is found to be dependent on the presence of K+ but not Na+, indicating that the Kv1.5 K+ channel maintained cation specificity when reconstituted on SSM.

In further specific embodiments of the solid supported membrane, the alkyl monolayer comprises a functionalized long-chain alkyl capable of bonding to the gold layer, and/or the functionalized alkyl comprises a long-chain alkyl thiol. The long-chain alkyl may be linear or branched and, in one embodiment, has a chain length of from about 8 to about 50 carbon atoms. In a more specific embodiment, the long-chain alkyl has a chain length of from about 10 to about 40 carbon atoms, and in a very specific embodiment the long-chain alkyl has a chain length of from about 12 to about 20 carbon atoms.

In one embodiment of the present solid supported membrane, the lipid monolayer comprises biological lipid, synthetic lipid, or combinations thereof. In a specific embodiment, the lipid monolayer comprises a Langmuir monolayer.

One embodiment of the present invention provides the solid supported membrane, wherein the chromium layer is formed on the support and the gold layer is formed on the chromium layer. In a specific embodiment the gold layer is reacted with the alkyl monolayer. The ion channel proteins may be modified to contain an SH group on an appropriately sized spacer arm, or may be attached covalently or non-covalently to bifunctional groups that react with both the protein of the ion channel and the solid support. These methods may be fully or partially automated by robotics. One embodiment provides that the functionally reconstituted ion channel is incorporated onto a surface of the solid supported membrane. In another embodiment, the functionally reconstituted ion channel is incorporated onto the bilayer.

Another embodiment of the present invention provides an assay for screening compounds to identify pharmacological agents affecting an ion channel. The assay comprises: (a) providing a solid supported membrane comprising: a support; a chromium layer; a gold layer; an alkyl monolayer, a lipid monolayer; and an ion channel protein, wherein the alkyl monolayer and the lipid monolayer form a bilayer, and further wherein a functionally reconstituted ion channel is supported; (b) contacting (a) with a solution comprising an ion; (c) applying a potential and obtaining a stable ion current; (d) contacting the solid support membrane with a compound to be identified; and (e) measuring a change in the ion current. In a specific embodiment, the assay further comprises a step of connecting an external electrode of a digitizer-amplifier to the solid supported membrane.

After formation of the solid-supported membrane comprising a functionally reconstituted ion channel, a potential is applied to the solid-supported membrane. In accordance with one embodiment of the present invention, potential is applied by connecting an external electrode of a digitizer-amplifier to the solid-supported membrane, although other techniques may be employed. For example, the gold layer may be connected to an external electrode of a digitizer-amplifier. In a further embodiment of the invention, the applied potential may be varied and direct electrical measurements can then be made on this system. One skilled in the art will appreciate the variation of the applied potential. For example, the potential may be varied from −80 to +80 mV.

In a further embodiment of the invention, the potential is applied after the ion channel is contacted with the compound to be identified or screened. One skilled in the art will appreciate the various methods for contacting an ion channel incorporated onto a solid-supported membrane with a compound to be identified or screened. The methods include, but are not limited to, contacting the compound with the ion channel in aqueous solutions of de-ionized water or physiological salt solutions.

The present synthetic membrane is particularly suited to use in rapid and high-throughput screening assays. Multiple wells may be formed on the surface of the bilayer. A suitable well enables a connection of the electrode with the bathing solution on the surface of the ion channel-containing membrane. Arrays or a single well may be formed using various techniques known in the art, including, but not limited to, etching, mechanical treatment, heating of the insulated area to achieve contact with the underlying substratum, or by the formation of a random set of holes in an insulating surface covering the face of the solid-supported membrane using a grid design for application of the insulating layer, and forming defects within the insulating area. Suitable defects may be formed by mechanical treatment in the insulating layer or heating. After formation of the various layers, the defects may be probed with a small electrode until a defect with suitable electrical properties is identified. All other defects within the well are then closed by application of an insulating material. The appropriate electrical properties for the defect area depends upon the membranes used, the ion channels to be studied, and the properties of the drugs to be screened.

The high mechanical stability of the solid-supported membrane allows the membrane to be reacted with multiple substances, washed and even re-used for multiple cycles. Due to this stability, the membrane can be larger than even planar lipid bilayers. For example, the solid-supported membrane may be of relatively large size encompassing literally hundreds of square micrometers for a membrane patch as compared to the conventional equivalent, typically encompassing approximately 1 square micrometer.

In accordance with a specific embodiment of the assay, the ion channel comprises a Kv1.5K⁺⁺ channel. In another embodiment, the assay is a high throughput assay wherein several compounds may be screened in parallel and/or in sequence. In a specific embodiment of the high throughput assay, further steps comprise: forming a plurality of wells on the surface of the bilayer; and distributing several compounds, one or more compounds per well. In another specific embodiment, the high throughput assay further comprises the steps of: washing the plurality of wells; and distributing additional compounds, one or more compounds per well. Some or all of the steps of the present assay may be automated.

The permissible large size of the solid-supported membrane allows for incorporation of many copies of the ion channel onto the surface of the solid-supported membrane. These ion channels may produce large currents that may be detected directly by single and inexpensive amplifiers connected with one or more of the wells formed on the surface of the solid-supported membrane. Ion channels have very high transport rates (billions of ions per sec) and the density of ion channels can be very high in simple expression/purification systems. Existing amplifiers are therefore easily configured to carry out the measurements of the present invention. A single amplifier and digitizer with commercially available software can handle up to 16 different membranes for current measurements lasting approximately 1 second each. Six parallel digitizer-amplifier systems can handle 96 samples (16×6 samples) in approximately 16 seconds. This rate allows high throughput screening of thousands of samples on each such system.

Furthermore, large libraries of compounds can be rapidly screened by noting whether mixtures of such compounds affect an electrical signal from an ion channel sensor device. If the mixture is without effect, other mixtures may be used. Therefore, it is possible to screen many compounds at once. Once it has been demonstrated that the mixture affects the compound, less complex mixtures may be prepared until the active compound is discovered. Alternatively, single compounds may be screened for an effect on an ion channel sensor that has been prepared in the wells.

The assay is particularly suitable for screening pharmacological agents that affect the ion channel by inhibiting the ion current through it.

Another embodiment of the present invention is directed to a method for forming a functionally reconstituted ion channel on a solid supported membrane. The method comprises the steps of: a. providing a solid support coated with a chromium layer and a gold layer; b. forming an alkyl-thiol monolayer on the gold layer; c. depositing a lipid monolayer on the alkyl-thiol monolayer to form a bilayer; and d. contacting the bilayer with an ion channel protein. In a specific embodiment of the method, the reconstituted ion channel comprises a Kv1.5K++ channel.

The use of solid-supported membranes containing ion channels have immense importance potential application in the field of environmental and smart sensors. Furthermore, the wide availability of literally hundreds of cloned ion channels and useful mutants, coupled with simple expression and purification systems, makes the present invention useful with many different systems. The synthetic membranes of the present invention are suitable for applications, including industrial applications, as bioelectric sensors.

The following example is intended to illustrate specific aspects and embodiments of the invention and should not be construed as limiting the invention as defined by the claims.

### EXAMPLE

The following example illustrates the preparation of a solid supported membrane comprising a functionally reconstituted Kv1.5 K+ channel. The experiment is designed to show that the reconstituted ion channel is functional, appropriately selective, and suitable for rapid and high-throughput screening of pharmacological compounds. Kv1.5 K+ channels are reconstituted on SSMs, and a current is measured in response to a holding potential. Several experiments are carried out to test the proposition that the current measured is Kv1.5-mediated and that thus functional reconstitution of Kv1.5 on SSM is achieved. Cation selectivity of the current and effects of specific Kv1.5 inhibitors on the current are examined. The reconstituted channel Kv1.5 currents are K+-dependent, virtually absent in the presence of Na+, and inhibited by specific Kv1.5 inhibitors. These effects were similar to those observed with Kv1.5 in biological membranes, supporting the view that Kv1.5 K+ channels are successfully reconstituted on SSM and maintain normal channel function. The strength and stability of SSM containing reconstituted functional ion channels suggests that it can be used to construct a screening device for pharmacological agents affecting ion channels.

1. Materials and Methods

1.1 Membrane Vesicle Preparation

An Ltk-cell line (mouse fibroblast cells) stably overexpressing Kv1.5 K+ channels under control of a dexamethasone promoter is used to prepare plasma membrane vesicles. Expression of Kv1.5 is induced in Ltk-cells by addition of dexamethasone to the medium. The dexamethasone-specific induction of channel expression is totally specific for Kv1.5 channels. Cells are grown in 2 μM dexamethasone for 24 hr prior to use. The cells are centrifuged for 5 min at 1000 rpm and resuspended in 1.0 ml of 20 mM HEPES (pH 7.5), 20 mM NaCl, 100 mM KCl, 1.0 mM EDTA, 0.02% NaN3, 1 mM PMSF, 10 μg/μl leupeptin, and 50 μg/μl aprotinin. After freeze-thawing twice, the membrane fragments/vesicles are collected following centrifugation at 12,000 g for 20 min at 4 oC. Membrane vesicles are also prepared from uninduced Ltk-cells (no dexamethasone incubation) transfected with Kv1.5 K+ channel cDNA and from non-transfected Lkt-cells.

1.2 Solid Supported Membranes (SSM) Preparation

Glass slides are first plated with chromium (5 nm), and then gold (150 nm). The slide is then immersed in ethanol containing 1% octadecanethiol (w/w) for 48 hr to attach alkyl thiol groups. After cleaning the gold plated slide with anhydrous isopropanol, epoxy resin is applied to the surface of the thiol-treated gold. Defects in the epoxy resin coating are used as the experimental chamber after coating the device with a lipid monolayer. A small area at the end of slide is left free of epoxy resin so that a silver wire may be soldered onto the surface of the gold plated slide. A 3:1 mixture of palmitoyl-oleoyl-phosphatidylserine (POPS) and pamitoyl-oleoylphosphatidylethanolamine (POPE) lipids, 10 mg/ml and 3.33 mg/ml in hexane respectively, is used to form a Langmuir monolayer, which is then deposited on the thiol-treated gold slide using the widely known Langmuir-Blodgett technique. The experimental wells are constructed by mounting a plastic ring on the surface with silicon grease and sealed with a coating of clear nail polish around the inner edge. A silver wire is soldered to the surface of gold plated slide to provide electrical connection.

1.3 Ion Channel Reconstitution and Current Measurement

Membrane vesicles containing Kv1.5 K+ channels are added to the lipid coated wells containing 125 mM KCl/10 mM K-HEPES pH 7.4. Currents are measured with an HS-2A headstage and Gene Clamp 500 amplifier (Axon Instruments). Channel currents are filtered at 60 Hz. Voltages ranging from −80 mV to +70 mV are applied in 10 mV increments for 200 msec, and electrical currents are recorded. Pclamp version 5.5 is used to acquire data and Clampfit 8.0 (Axon Instruments) is used to compare current recordings. Similar measurements are made using membrane vesicles isolated from non-transfected Lkt-cells and from transfected Ltk-cells that had not been induced to express Kv1.5 with dexamethasone. Cation selectivity is measured by removing K+ from the medium and replacing it with Na+. Currents are first measured with K+ present. SSMs are then washed with K+-free, Na+-containing medium and currents were measured with Na+ present. Currents with K+ present are re-measured. Statistical analysis is carried out using the students t-test.

1.4 Whole Cell Patch Clamp Electrophysiology

Whole-cell Kv1.5 current recordings are made at room temperature via the gigaseal patch clamp technique using an Axopatch-1D amplifier (Axon Instruments, Foster City, Calif.). Ltk-cells overexpressing Kv1.5 channels are cultured for 24-72 hr, induced to express Kv1.5 channels by 24 hr incubation with 2 μM dexamethasone prior to use for patch clamp studies. Small, spherical cells approximately 10 microns in diameter are used for all patch recordings. Electrodes are made from TW-150F glass capillary tubes (World Precision Instruments, New Haven Conn.) and had resistances of 1.5-3.0 megohms when filled with internal solution containing: 110 Mm KCl, 5 mM K2ATP, 5 mM K4BAPTA, 1 mM MgCl2 and 10 mM HEPES, adjusted to pH 7.2 with KOH. The external solution contained: 130 mM NaCl, 4 mM KCl, 1.8 mM CaCl2 1 mM MgCl2, 10 mM HEPES, 10 mM glucose, adjusted to pH 7.4 with NaOH. Series resistance is compensated following rupture of the seal. Currents are sampled at 1 KHZ and filtered at 500 Hz. Cells are pulsed to +60 mV every 5 sec from a holding potential of −70 mV in 20 mV increments. After stable control currents are obtained, inhibitors are perfused onto the cells at increasing concentrations until maximal inhibition is obtained for a given concentration. Whole cell patch data are analyzed using Clampfit 8.0 in pCLAMP software (Axon Instruments). IC50 values for compounds are determined by nonlinear regression analysis using GraphPad Prism software (San Diego, Calif.).

1.5 Physical Characterization

A current is recorded using K+ medium in the absence of any added membrane vesicles at −80 mV for each well on the SSM surface. The surface area not covered by epoxy is determined mathematically by using a microscope and scale. The relationship between current and SSM area is determined.

1.6 Materials

Pre-cleaned glass slides are obtained from Becton Dickenson Labware. Gold coating is performed by H.L. Clausing, Inc. (Skokie, Ill.). Silver wire, 1-octadecanethiol and DMSO are from Aldrich (Milwaukee, Wis.). HEPES, KCl, and NaCl are from Sigma (St. Louis, Mo.). POPS and POPE are from Avanti Polar Lipids and dissolved in reagent grade n-hexane. Ag/AgCl reference electrode is obtained from Warner Instrument, Corp (New York, N.Y.). Epoxy resin (5 min epoxy, No. 14250) is from Devcon. The inhibitors used are from Procter and Gamble Pharmaceuticals (Cincinnati, Ohio). Compound A, prepared according to the procedure provided in U.S. Pat. No. 6,083,986, is (N-[(2S,3S)-3-[[(4-ethylphenyl)sulfonyl]amino]-2,3-dihydro-2-hydroxy-1H-inden-5yl]-3-methoxybenzamide). Compound B, prepared according to the procedure provided in U.S. Pat. No. 6,174,908, is (2-(3,4-dimethyphenyl)-3-[2-(4-methoxyphenyl)ethyl]-thiazolidin-4-one.

2. Results

2.1 Reconstitution of Kv 1.5 into a SSM and Cation Selectivity

Before addition of membrane vesicles, the current across the SSM is measured at different holding potentials and plotted as an I/V curve. Membrane vesicles containing dexamethasone-induced Kv1.5 K+ channels are then added to the SSM, and after approximately 20 min, an increase in the current is evident at the same potentials. Typical current recordings of SSM without membrane vesicles and with membrane vesicles containing induced Kv1.5 K+ channels are generated. At −80 mV, the increase is 7.76±3.10 (n=6) μA and at the +70 mV, the increase is 8.06±3.18 (n=6) μA. These currents are significantly (P<0.01) higher than the currents measured in the absence of membrane vesicles, but are not significantly different in magnitude from each other. The INV relationship is not linear and shows similar rectification at both positive and negative holding potentials. Currents generated using membrane vesicles from Kv1.5 transfected Ltk-cells±dexamethasone as well as from non-transfected Ltk-cells are compared. The current measured across SSM without any membrane vesicle addition (leak current) is subtracted from the current measured in the presence of membrane vesicles. Using membrane vesicles expressing dexamethasone-induced Kv1.5 K+ channels, a large current (20.11±3.55 μA, n=4) is measured which is virtually absent when membrane vesicles from non-transfected Lkt-cells are used (1.90±1.27 μA, n=4). This difference is highly significant (P<0.005). When membrane vesicles prepared from uninduced Kv1.5 transfected Lkt-cells (not incubated with dexamethasone) are added to the SSM, a very small current increase of 4.88±1.66 μA (n=8) is observed. This current is also significantly lower (P<0.01) than that measured with membrane vesicles containing dexamethasoneinduced Kv1.5 K+ channels and is not significantly different from that measured using membrane vesicles from non-transfected Lkt-cells.

To investigate whether the increased current is occurring through Kv1.5 K+ channels successfully and functionally reconstituted on the SSM, cation selectivity is examined. The effect of K+ removal and replacement with Na+ on the current measured at −80 mV is investigated. Current is measured first with K+ present and then with Na+ present. When K+ is removed from the medium, the current decreased significantly (P<0.01) from 20.11±3.55 (n=4) to 2.73±1.56 (n=4) μA. Using membrane vesicles from non-transfected Lkt-cells in which Kv1.5 channels are absent, currents are low and similar whether in KCl or NaCl 11 medium. These findings indicate that Kv1.5 K+ channels reconstituted on the SSM are functional and highly selective for K+ over Na+.

2.2 Effect of Kv1.5 K+ Channel Inhibitors

To further support the view that the measured current is due to the presence and function of Kv1.5 K+ channels on the SSM, and not due to a non-specific leak in the SSM, the effect of 2 specific inhibitors of Kv1.5 K+ channels, compounds A and B, is measured. The effect of 100 nM compound A on the current is measured at varying holding potentials with Kv1.5 K+ channels reconstituted on the SSM. The current decreased over the range of holding potentials outside of the range of ±20 mV. The effect of varying concentrations of compound A on Kv1.5 K+ channel current is expressed as fractional inhibition (I/Imax). Compound A dose-dependently inhibits the Kv1.5 K+ channel current at -80 mV with half-maximal inhibition, IC50=218 nM (n=3). Similar inhibition of Kv1.5 K+ channel currents at −80 mV is observed with compound B with IC50=265 nM (n=3). FIGS. 5A and 6A show Kv1.5 K+ channel currents recorded by whole cell patch clamp of dexamethasone-induced transfected Lkt-cells without (control) and with 300 nM of inhibitor compounds A and B respectively. A similar level of inhibition of the current is observed with both compounds. Using whole cell patch clamp, IC50 was 170 nM for Compound A and 137 nM for Compound B, values similar to those calculated from experiments using Kv1.5 K+ channels incorporated into SSMs. Stability and robustness of the SSM-mounted Kv1.5 K+ channels and reversibility of the inhibitor effects is also examined. A typical experiment in which current recordings are carried out over a period of 3.5 hrs is conducted. The effect of 774 nM compond A is tested and retested 3 times after washing the SSM with medium. A DMSO control is also carried out. 774 nM compound A caused similar current inhibition each time it was tested and currents measured after washes are similar to the first current recording before inhibitor is added. No deterioration of currents is observed over 3.5 hrs. DMSO has no effect. Thus SSM mounted Kv1.5 K+ channels are very stable. Inhibitor effects are reversible and the SSM with Kv1.5 K+ channels may be re-used.

2.3 Physical Characterization

A current is associated only with the lipid coating without any membrane vesicles present. These leak currents are measured and plotted against the corresponding SSM area. The diameter of the area varies from 200-700 μm as estimated by microscopic examination. There was moderate correlation of the SSM current (without membrane vesicles) with area of the SSM as estimated microscopically. For each individual lipid bilayer used for an experiment, this leak would be a constant related only to the area of the bilayer. Under ideal conditions, the lipid monolayer obtained with the Langmuir-Blodgett technique is tightly packed resulting in high capacitance. Evidence of leak current is present. In the experimental setup used, the amount of insulation achieved with a lipidcoating is proportional to the area.

3. Discussion

Voltage gated Kv1.5 K+channels are reconstituted on solid supported membranes (SSMs) as indicated by an increased K+ current upon the addition of membrane vesicles contained Kv1.5 K+ channels. A reduction in the K+ current is also demonstrated with addition of inhibitors to Kv1.5 K+ channels as well as replacement of K+ with Na+. The IC50 values for the inhibitors using Kv1.5 reconstituted on SSM are comparable to their patch clamp equivalent (15, 16) The SSM reconstituted Kv1.5 channels responded to the inhibitors as if they were in a biological membrane. The potential advantages of using SSM mounted ion channels include their mechanical and physical stability compared to that of unsupported membranes, which are not easily washed and re-used, and the system is amenable to automation. The testing wells containing SSM mounted ion channels may be simply washed with buffer and reused many times. Hence, this system is adaptable for rapid screening of pharmacological compounds. The inhibitor effects on Kv1.5 K+ currents appear to be hyperbolic, suggesting a simple bimolecular interaction between the drugs and the channel, without effects on the membrane itself Effects on the membrane per se would be expected to be linear, rather than hyperbolic. This essentially rules out an effect of the compounds on the SSM lipids. It is known that Kv1.5 K+ channels in cells are rectified. However, the I/V relationship indicates that ions move at both positive and negative holding potentials. This behavior may be explained by assuming that the orientation of Kv1.5 was a mixture of inside-out and outside-in Kv1.5 channels. The resultant channel current would be the sum of the activity of the channels in both orientations and thus would show rectification at positive and negative holding potentials. This is likely the case since Kv1.5 K+ channels are introduced to the SSM surface as membrane fragments. Currents observed with this method are in the μA range whereas typical single channel currents are in the pA range. There are many active Kv1.5 ion channels on the surface of the SSMs. The ion currents measured are large (the sum of those occurring through the channel proteins in the SSM), as expected from the large surface area of the SSM.

There are reports of water being present between a bilayer film and a solid support, where water exists as a thin layer of 10-20 Å. A theoretical model introduced by Sparr et al reveals that membranes are permeable to water under certain conditions, which may be similar to the SSM conditions.

In summary, reconstitution of ion channels on solid supported membranes is demonstrated. A low leak current increases when Kv1.5 K+ channels are introduced on the SSM surface. This is indirect evidence that ion channels are reconstituted. Moreover, the level of the K+ current may be reduced by removal of K+ (and replacement with Na+) or by addition of ion channel inhibitors. The IC50s obtained using SSMs are comparable to those obtained from patch clamp studies. Kv1.5 K+ channels reconstituted on SSMs maintain cation specificity, as seen in cellular systems. Ion channels mounted on SSMs can effectively substitute for the time-consuming patch clamp method, enabling rapid screening of pharmacological reagents.

What is claimed is:

1. A solid supported membrane comprising: a support; a chromium layer; a gold layer; an alkyl monolayer, a lipid monolayer; and an ion channel protein, wherein the alkyl monolayer and the lipid monolayer form a bilayer, and further wherein a functionally reconstituted ion channel is supported.

2. The solid supported membrane as recited in claim 1, wherein the ion channel protein comprises native protein, recombinant protein, or combinations thereof

3. The solid supported membrane as recited in claim 1, wherein the reconstituted ion channel comprises a Kv1.5K⁺⁺ channel.

4. The solid supported membrane as recited in claim 1, wherein the alkyl monolayer comprises a functionalized long-chain alkyl capable of bonding to the gold layer.

5. The solid supported membrane as recited in claim 4, wherein the functionalized alkyl comprises a long-chain alkyl thiol.

6. The solid supported membrane as recited in claim 4, wherein the long-chain alkyl may be linear or branched and has a chain length of from about 8 to about 50 carbon atoms.

7. The solid supported membrane as recited in claim 6, wherein the long-chain alkyl has a chain length of from about 10 to about 40 carbon atoms.

8. The solid supported membrane as recited in claim 7, wherein the long-chain alkyl has a chain length of from about 12 to about 20 carbon atoms.

9. The solid supported membrane as recited in claim 1, wherein the lipid monolayer comprises biological lipid, synthetic lipid, or combinations thereof.

10. The solid supported membrane as recited in claim 1, wherein the lipid monolayer comprises a Langmuir monolayer.

11. The solid supported membrane as recited in claim 1 wherein the support comprises agar polymers, conducting polymers, glass, silinated glass, aklylated hydrogels, plastics, MYLAR, metallic substrates, or combinations thereof.

12. The solid supported membrane as recited in claim 11, wherein the support comprises silinated glass.

13. The solid supported membrane as recited in claim 1, wherein the chromium layer is formed on the support and the gold layer is formed on the chromium layer.

14. The solid supported membrane as recited in claim 1, wherein the gold layer is reacted with the alkyl monolayer.

15. The solid supported membrane as recited in claim 1, wherein the functionally reconstituted ion channel is incorporated onto a surface of the solid supported membrane.

16. The solid supported membrane as recited in claim 1, wherein the functionally reconstituted ion channel is incorporated onto the bilayer.

17. An assay for screening compounds to identify pharmacological agents affecting an ion channel, the assay comprising:
(a) providing a solid supported membrane comprising: a support; a chromium layer; a gold layer; an alkyl monolayer, a lipid monolayer; and an ion channel protein, wherein the alkyl monolayer and the lipid monolayer form a bilayer, and further wherein a functionally reconstituted ion channel is supported;
(b) contacting (a) with a solution comprising an ion;
(c) applying a potential and obtaining a stable ion current;
(d) contacting the solid support membrane with a compound to be identified; and
(e) measuring a change in the ion current.

18. The assay as recited in claim 17, further comprising a step of connecting an external electrode of a digitizer-amplifier to the solid supported membrane.

19. The assay as recited in claim 17, wherein the ion channel comprises a Kv1.5K⁺⁺ channel.

20. The assay as recited in claim 17, wherein the assay is a high throughput assay wherein several compounds may be screened in parallel and/or in sequence.

21. The high throughput assay as recited in claim 20, further comprising the steps of forming a plurality of wells on the surface of the bilayer; and distributing several compounds, one or more compounds per well.

22. The high throughput assay as recited in claim 21 further comprising: washing the plurality of wells; and distributing additional compounds, one or more compounds per well.

23. The assay as recited in claim 20, wherein at least some steps of the assay are automated.

24. The assay as recited in claim 17, wherein the identified pharmacological agents affect the ion channel by inhibiting the ion current through it.

25. A method for forming a functionally reconstituted ion channel on a solid supported membrane, the method comprising the steps of:
a. providing a solid support coated with a chromium layer and a gold layer;
b. forming an alkyl-thiol monolayer on the gold layer;
c. depositing a lipid monolayer on the alkyl-thiol monolayer to form a bilayer; and
d. contacting the bilayer with an ion channel protein.

26. The method as recited in claim 26, wherein the reconstituted ion channel comprises a Kv1.5K++ channel.

27. A bioelectric sensor comprising the solid supported membrane as recited in claim 1.
